(19) United States
(12) Patent Application Publication
(10) Pub. No.: US 2002/0032435 A1
(43) Pub. Date: Mar. 14, 2002

(54) Tracking surgical implements with integrated circuits

(76) Inventor: Bruce H. Levin, Marion Station, PA (US)

Correspondence Address: KENYON & KENYON, 1500 K STREET, N.W., SUITE 700, WASHINGTON, DC, 20005, US

(21) Appl. No.: 09/883,991

(22) Filed: Jun. 20, 2001

Related U.S. Application Data
(60) Provisional application No. 60/212,680, filed on Jun. 20, 2000.

(51) Int. Cl. A61B 17/00; A61N 1/30; A61B 19/00

(52) U.S. Cl. 606/1; 604/19; 128/899

(57) ABSTRACT

The present invention provides devices, methods, and systems that monitor and track medical products, including surgical implements. The device can be a surgical implement including at least one integrated circuit that uniquely identifies the surgical implement by a unique identifier is provided. One method for monitoring and tracking surgical implements includes identifying at least one surgical implement including an integrated chip, where each surgical implement is uniquely identified. Systems for monitoring and tracking surgical implements are also provided. One system includes at least one surgical implement, each surgical implement including an integrated circuit that stores a unique identifier of the surgical instrument and a detector that detects the surgical implement by detecting the unique identifier from the integrated circuit. Labels for labeling medical products are also provided.

### FIELD OF THE INVENTION

The present invention relates to devices, labels, methods, and systems to monitor and track medical implements and products containing integrated circuits. Specifically, embodiments of the present invention relate to preventing these medical implements from being inadvertently left within a human or animal following completion of medical procedures. In addition, embodiments of the present invention are meant to decrease errors resulting from sub-optimal production, processing, distribution, and administration of medical products, including but not limited to pharmaceuticals and blood products.

### BACKGROUND OF THE INVENTION

During surgery it is necessary to place surgical implements, such as sponges, scalpels, needles, gauzes, and the like near or into a wound cavity. Even though thorough manual counts are conducted following the completion of surgery, this method is time consuming, tedious and error prone. Indeed, surgical implements are too frequently left inside patients resulting in complications including trauma, pain, infection or death.

A number of conventional methods exist to make sure that all surgical implements have been removed from a patient, but all have drawbacks. The most well known method is to use X-rays. In this procedure, the surgical implements have radio opaque material embedded within them. Following the completion of surgery and suturing of the patient, an X-ray machine is moved over the patient and an X-ray is taken of the wound area to determine whether radio opaque materials are present in the patient. However, some materials may be too small to be easily seen on X-ray, or they may be otherwise obscured by bone or tissues within radio dense areas. If any surgical implements are found on the X-ray within the sutured area, then the patient is reopened to retrieve the retained materials. This way, implements left within a patient are removed. However, each time this procedure is performed, expensive operating room time is wasted and other patients may have their surgeries delayed. Furthermore, the patient is subjected to more anesthesia time and otherwise unnecessary radiation.

Another method suggested by U.S. Pat. No. 4,193,405 to Abels, detects a radio-frequency (“RF”) transponder embedded in a surgical sponge. In this method, tagging of surgical articles with ferrite or other semiconductor material is done such that when they are exposed to two selected frequencies the material will resonate. This resonance can then be detected by a RF receiver. However, this method merely relates to a transponder, no data is recorded as to type of object, time rank of object, nor does it allow for master categorization which would alert the user that an object is in fact missing, even in the absence of a detected failure. Hence, this level of safety is easily breached.

In U.S. Pat. No. 4,658,818 to Miller, a miniature battery-powered oscillator is attached to each surgical implement and activated prior to its initial use. The output of each oscillator is in the form of a low powered pulse which is coupled to the body's fluids and tissue. After the surgery is completed, but prior to suturing, a detection system is used to sense for any pulses generated within the body. However, this system also does not provide information as to object type, rank timing or master categorization, and merely serves as a pulse alarm.

Another system that has recently been devised is disclosed in U.S. Pat. No. 5,931,824 to Stewart. This system is drawn to placing machine-readable information on individual surgical sponges. In addition, each sponge has X-ray detectable material embedded within it. This system requires that each sponge is scanned which is tedious, and allows for neither non-orientational registration nor perimeter scanning.

Additionally, sub-optimal logistics result in medication and other errors, which have resulted in significant morbidity and mortality.

### SUMMARY OF THE INVENTION

The present invention provides devices, methods, and systems that monitor and track medical materials, including surgical implements.

In an embodiment of the present invention, a surgical implement including at least one integrated circuit that uniquely identifies the surgical implement by a unique identifier is provided.

In another embodiment of the present invention, a method for monitoring and tracking surgical implements is provided. The method includes identifying at least one surgical implement including an integrated chip, where each surgical implement is uniquely identified. Another method of the present invention provides for monitoring and tracking medical materials. This method includes uniquely identifying at least one medical material by a unique identifier, each medical material including at least one integrated circuit having the unique identifier programmed therein and monitoring each medical material by its unique identifier. In another embodiment of the present invention, a method for monitoring surgical implements in conjunction with a surgical procedure is provided. The method includes initializing at least one surgical implement where each surgical implement includes an integrated circuit, registering the surgical implement prior to a surgical procedure by programming a unique identifier in the integrated circuit, and accounting for the surgical implement at the completion of the surgical procedure by receiving the unique identifier from the surgical instrument.

The present invention also includes systems. In one embodiment of the present invention a system for monitoring and tracking surgical implements is provided. The system includes at least one surgical implement, each surgical implement including an integrated circuit that stores a unique identifier of the surgical instrument and a detector that detects the surgical implement by detecting the unique identifier from the integrated circuit. Another embodiment of the present invention includes a system for monitoring and tracking surgical implements including at least one surgical implements, including at least one integrated circuit and a sensor for sensing the surgical implements based on a signal received from each integrated circuit. In another embodiment of the present invention, a system for monitoring surgical implements used in conjunction with a surgical procedure is provided. This system includes at least one surgical implement comprising an integrated circuit, the integrated circuit associating a unique identifier with each of the surgical implements and emitting a signal containing the unique identifier, a detector that detects the signal emitted by the surgical implement, and an output device to process information provided by the detector. The present invention also provides another embodiment of a system, including at least one surgical implement comprising an integrated circuit, the integrated circuit associating a unique identifier with each of the surgical implements and emitting a signal containing the unique identifier, a platform with a detector that detects the signal and determines a placement and removal of each of the surgical implements from the platform based on the detected signal, and an output device that receives and processes information provided by the detector. Another embodiment of the present invention provides a system for monitoring patients including at least one medical material, each medical material including a first integrated circuit, at least one patient identification tag, each patient identification tag including a second integrated circuit, and a sensor that monitors the medical materials and patient identification tags based on signals received from the first and second integrated circuits.

The present invention also provides a medical label including at least one integrated circuit, where the integrated circuit uniquely identifies a medical product the medical label is attached to. In another embodiment of the present invention, a blood product label is provided, which includes a label attached to a blood product, the label including at least one integrated circuit that uniquely identifies the blood product. The present invention also provides a blood product container including the blood product label. Finally, the present invention provides medical product including at least one integrated circuit that uniquely identifies the medical product by a unique identifier.

### BRIEF DESCRIPTION OF THE DRAWINGS

FIG. 1 shows a block diagram of the sensor system and two integrated circuits to be used in surgical implements.

FIG. 2 shows a block diagram of the sensor system and two integrated circuits to be used in surgical implements.

FIG. 3 shows an embodiment of a database table for the sensor system.

FIG. 4 shows an embodiment of a database table for the sensor system.

FIG. 5 shows a flow chart of registration instructions for the sensor system.

FIG. 6 shows an embodiment of the sensor system in a patient ID bracelet and integrated circuits in blood bags and syringes.

### DETAILED DESCRIPTION

Embodiments of the present invention relate to methods, devices, labels, and systems for monitoring medical implements products containing integrated circuits, microchips, or Radio Frequency Ids (RFID). Prior to a medical procedure, each of the implements to be used is registered with a sensor system such that the implement is uniquely identified. Following the medical procedure, each of the implements that was registered is then accounted for.

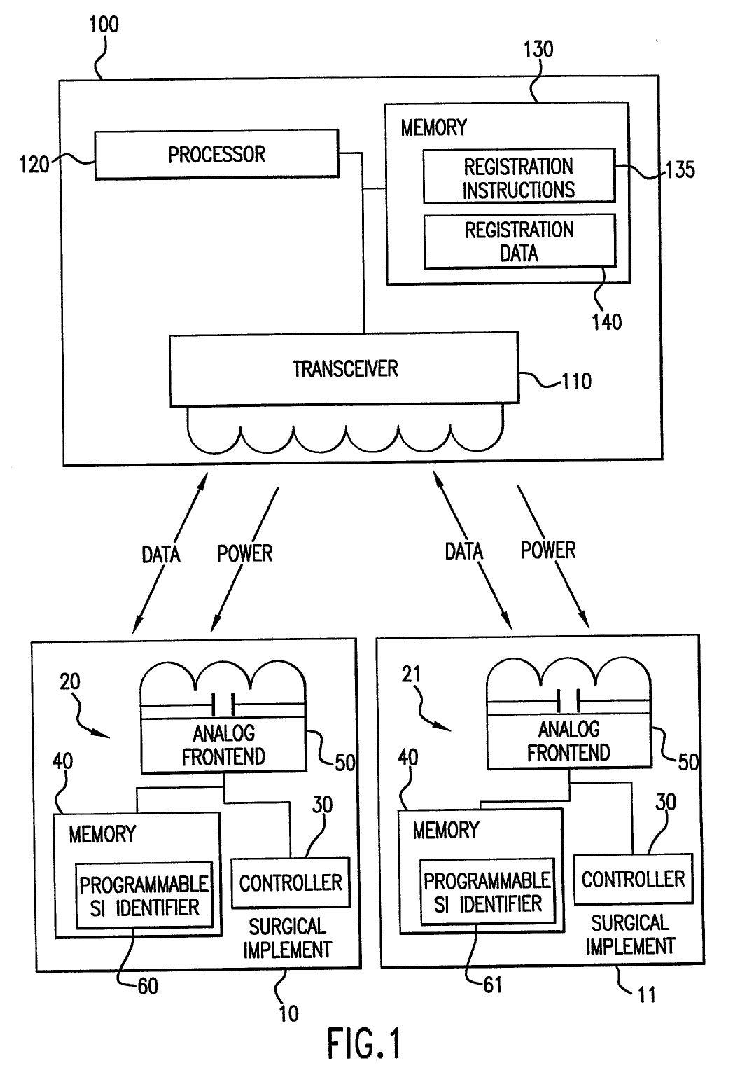

FIG. 1 shows an example of one embodiment of the present invention. Like elements are labeled with like numbers. In FIG. 1, two surgical implements 10 and 11 are shown and a sensor system 100. Surgical implements, as used herein, include, but are not limited to, sponges, needles, scalpels, gauze, forceps, and scissors and the like.

Also, the scope of the term surgery or surgical is not to be limited, but should include all types of medical procedures and is used herein interchangeably with the term medical.

In FIG. 1, surgical implement 10 includes an integrated circuit 20, and surgical implement 11 includes an integrated circuit 21. The integrated circuit 20 includes an analog front-end 50, which could, for example, be a LC circuit; a memory 40; and a controller 30. In the memory 40 of surgical implement 20 there can be stored a programmable surgical implement identifier 65. This programmable surgical implement identifier is used as a unique identifier for each surgical implement. This particular illustration is but one example of how the present invention could be practiced and is not meant to limit the scope in any way.

The integrated circuits 20 and 21 are powered through radio frequency (“RF”) signals generated by the sensor system 100. However, the integrated circuits may also be powered by any known source of energy, including, but not limited to, a battery, exposure to air, moisture, certain chemicals or substances, changes in temperature, pH, or motion. Additionally, the integrated circuits may be powered by induction, EMF, other radiation or by the potential, chemical, or electrical gradients, or microelectric currents of the body.

The integrated circuits 20 and 21 are encapsulated in plastic and then incorporated into surgical implements. Generally, the integrated circuits are incorporated into each of the different surgical implements or materials natively. Therefore the integrated circuits are incorporated in such a way as to be encapsulated, hermetically sealed, flexible, heat, shock and water resistant and sterilized or sterilizable. The integrated circuits are also incorporated in a manner that does not impede or hinder the normal function of the medical implement. Because the surgical implements include many different instruments, incorporation of the integrated circuits into each different implement needs to be individualized to that implement and this can be done by those of skill in the art. Also, the integrated circuits can be incorporated into or structurally associated with x-ray opaque material.

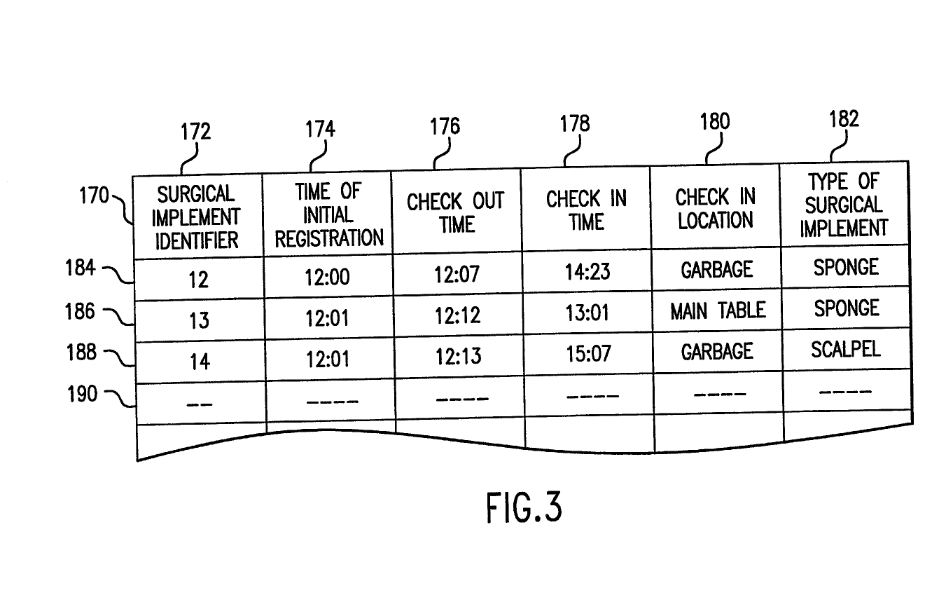

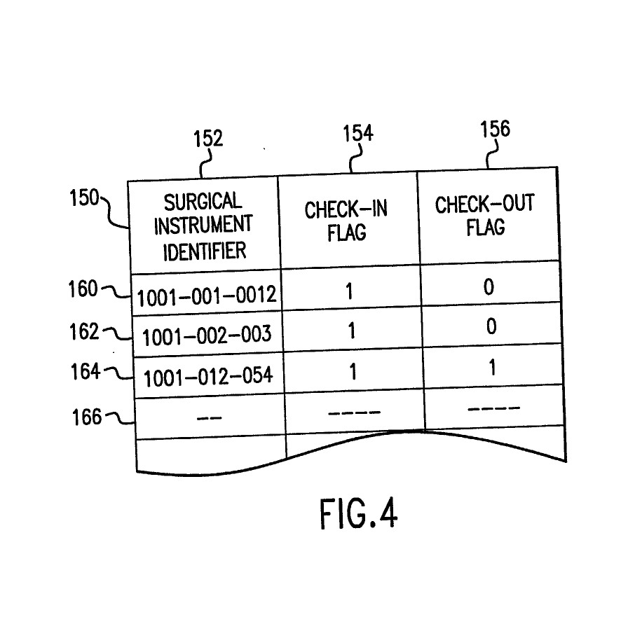

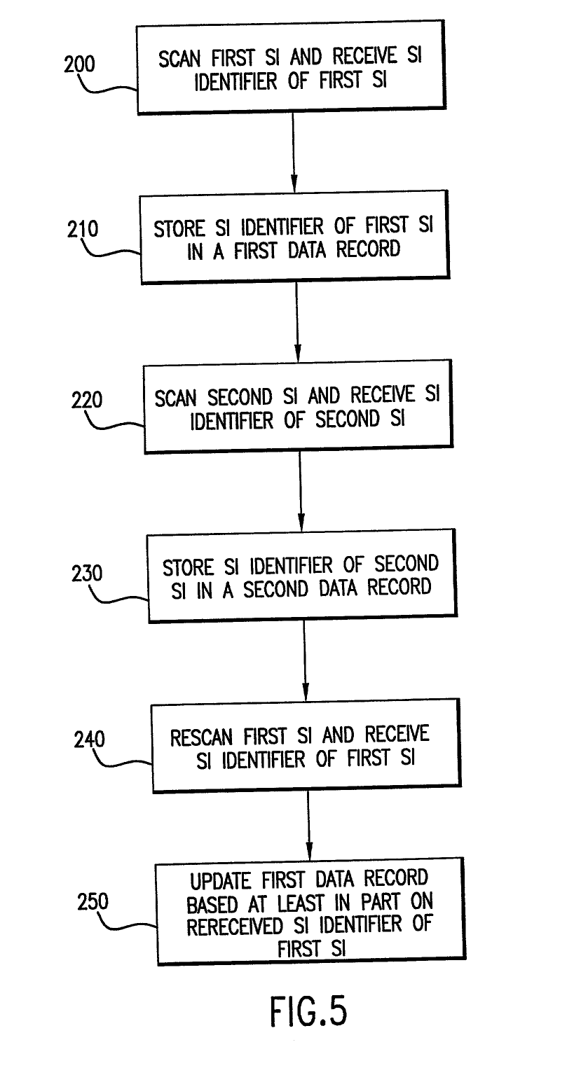

FIG. 1 also shows a sensor system 100. The sensor system 100 includes a processor 120, a memory 130, and a transmitter 110. The memory 130 of the sensor system includes registration instructions 135 and registration data 140. The processor 120 can be a Pentium® III manufactured by Intel of Santa Clara, Calif., an Application Specific integrated circuit (“ASIC”), a microcontroller, etc. The registration instructions 135 will be explained more fully with reference to FIG. 5 and the registration data 140 will be explained more fully with reference to FIG. 3 and FIG. 4. The sensor system 100 may also include an interface consisting of a computer terminal or terminals (not shown). In addition, there may be additional auxiliary sensory systems used in conjunction with the main sensor system throughout an operating room. Operating room as used herein, includes, but is not limited to, an operating theater, an operating room, an operating suite, or any other room where surgery or any invasive procedure of any type is performed on humans or animals.

One example of an integrated circuit and corresponding base station that a person of ordinary skill in the art could use to practice the present invention is TEMIC Semiconductors TK5552 transponder integrated circuit and base station, as described in TEMIC Semiconductors, “TK5552”, Rev. A4, Apr. 26, 2000, which is hereby incorporated by reference, in its entirety. TEMIC Semiconductors' TK5552 integrated circuit transponder is a programmable read/write transponder with an operation range of up to 10 cm powered by a RF field generated by the base station.

Other embodiments of the integrated circuit can be made of molecular switches using nanotubes as wires, such as described by Rotman in “Molecular Computing” Technology Review 103: 52-58 (May-June 2000), or molecular conductors such as benzine dithol as described by Reed et al. in “Computing with Molecules” Scientific American, 282: (June 2000), both of which are hereby incorporated by reference in their entirety.

In addition, the integrated circuit can be a RFID. The RFID may be readable only or readable and writeable. One example of an RFID that could be used in the present invention is disclosed in U.S. Pat. No. 6,249,227, hereby incorporated by reference, in its entirety.

Embodiments of the present invention relate to tracking and monitoring surgical implements. To that end, as can be seen in FIG. 1, data is read and written to and from the sensor system 100 and integrated circuits 20 and 21. The sensor system 100 assigns the programmable surgical implement identifier 60 to the surgical implement 10 and surgical implement identifier 61 to surgical implement 11 while collecting various data to compile the registration data 140 in the sensor system 100 and memory 130.

An example set of registration instructions 135 stored in the memory 130 of the sensor system 100 is shown in FIG. 5. In the first step 200, the sensor system 100 scans a first surgical implement and receives the surgical implement identifier of the first surgical implement. At step 210, the surgical implement identifier of the first surgical implement is stored in the registration data 140 in the memory 130 of the sensor system 100 in a first data record. In step 220 the sensor system 100 scans a second surgical implement and receives a surgical implement identifier of the second surgical implement. At step 230 the surgical implement identifier of the second surgical implement is stored in a second data record in the registration data 140. In step 240 the sensor system 100 re-scans the first surgical implement and re-receives the surgical implement identifier of the first surgical implement. In step 250 the first data record is updated based at least in part on the re-received surgical implement identifier of the first surgical implement.

The registration data 140 can be a relational database 170 shown in FIG. 3. Database 170 includes records 184-190, which are accessible using a suitable database management system software. Each record 184-190 of database 170 contains six fields 172-182. Field 172 holds the surgical implement identifier, which can be any unique identifier, for example a number(s), letter(s), a combination of numbers and letters, a frequency, or the like. In this embodiment, the memory 40 of the integrated circuit 20 is programmable, so the surgical implement identifier 60 is programmable. Therefore, field 172 can be programed by the sensor system. Field 174 indicates the initial time of registration, for example when the sensor system first senses the surgical implement and is associated with a registration identifier. Field 176 indicates when the given surgical implement was checked out to be used in a surgery and is associated with a checked-out identifier. Field 178 holds information about when the given surgical implement was checked back in following it use and is associated with a checked-in identifier. Field 180 holds information about the check-in location within the operating room and field 182 indicates what the actual surgical implement is, for example, a sponge, a scalpel, gauze, or the like. This particular arrangement of fields is but one illustration of how the invention may be practiced. For example, certain fields can be omitted, additional fields can be provided, or the arrangement of fields can be changed. For example, additional fields for the check-in or check-out location can be added. Also, a field could be added that indicated the count of each implement. For example, that a particular sponge was sponge five of twenty-5/20 or that a scalpel was two of five-2/5.

Each record 184-190 of database 170 associates a surgical implement identifier with time of check-out and time of check-in. In addition, other information is associated with each surgical implement, for example, the actual surgical implement and the location of its check-in. By compiling this information it becomes possible to monitor each individual surgical implement.

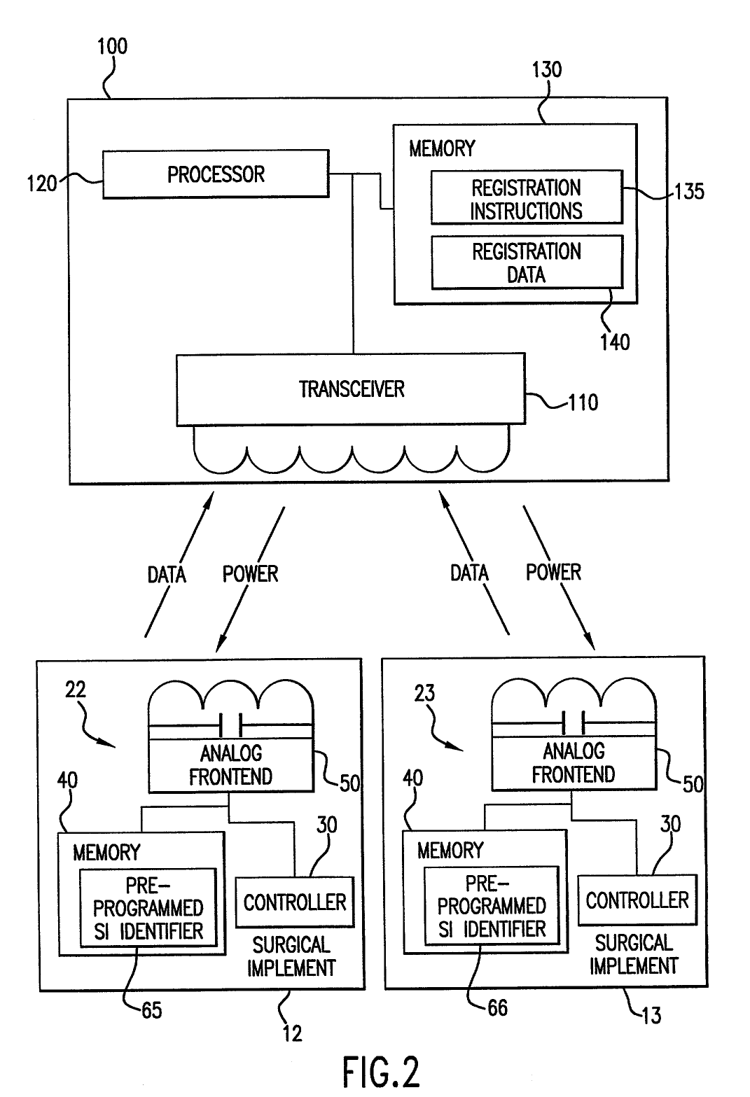

FIG. 2 shows a similar embodiment as FIG. 1, except that the data is only shown being read by the sensor system 100. The memory 40 of the integrated circuit 22 has a pre-programmed surgical implement identifier 65 as compared to the programmable surgical implement identifier 60 of FIG. 1, and integrated circuit 23 has a pre-programmed surgical implement identifier 66.

FIG. 4 shows database 150, which could be used with the embodiment of the present invention shown in FIG. 2. Database 150 includes records 160-166, which are accessible using a suitable database management system software. Each record 160-166 of database 150 contains three fields 152-156. Field 152 contains the surgical implement identifier, which is pre-programed in the surgical implement. The pre-programed identifier could be programed, for example, in such a way as to indicate the hospital, the type of implement, the number of the implement, or other parameters desired to be associated with the implement. This particular programing is one illustration of how the invention may be practiced. Field 154 corresponds to a check-in “flag” if the surgical implement has been taken to be used, while field 156 corresponds to a check-out “flag” when the surgical implement is brought back after being used. This is a simplified version of the database shown in FIG. 3.

Prior to surgery, each surgical implement having an integrated circuit in it is placed on or near the main sensor system. The sensor system assigns an individual surgical implement identifier to each surgical implement and records initial data (e.g., initial time of registration). In order to make sure that no unregistered implements are located within the operating room, the sensor system will note all incomplete implement integrated circuit data profiles and alert upon such sensing. When the surgery begins and the surgical implements are used, the sensor systems records the time each surgical implement is checked-out/used. When the surgical implement is done being used and the surgical implement is replaced either on or near the main sensor system or in an auxiliary sensory system, the time and optionally, the location, of check-in for each surgical implement is recorded. Following surgery, a comparison is completed of surgical implements checked-out and surgical implements checked-in and a list is generated to identify which surgical implements are missing, if any. An output device, such as a computer can be used to display the list. In addition, an alarm will sound if any surgical implements are checked-out but not checked back in from the sensor or the output device. Alternatively, the sensor system can keep a running comparison of the surgical implements that have been checked-out and the ones checked-in. In this manner the sensor system can be programmed to alert at particular times during the procedure in order to track the surgical implements throughout the procedure.

The functions of the sensor system include, but are not limited to, sensing, tracking, marking, managing, monitoring, setting, controlling, checking, dating, timing, billing inventory control and comparing with protocol. When the implements are placed on, in, or near the main or auxiliary sensor system, each is detected and assigned a unique and individual identifier by the associated sensor system. The identifier used herein includes, but is not limited to, information regarding the product, numbers, strings of letters and numbers, strings of letters or other codes, or a frequency. The sensor system and the auxiliary sensor systems as used herein include, but are not limited to, handheld devices, perimeter systems, entry/exit systems, tables, trays, shelves or stands.

In another embodiment, a backup system could be incorporated into the surgical implements using a second integrated circuit, or tag, which would generate an error message when read by a sensor system if there was a problem with a primary integrated circuit.

In another embodiment, the initial assigning of surgical implement identifiers is performed when the surgical implements enter the operating room.

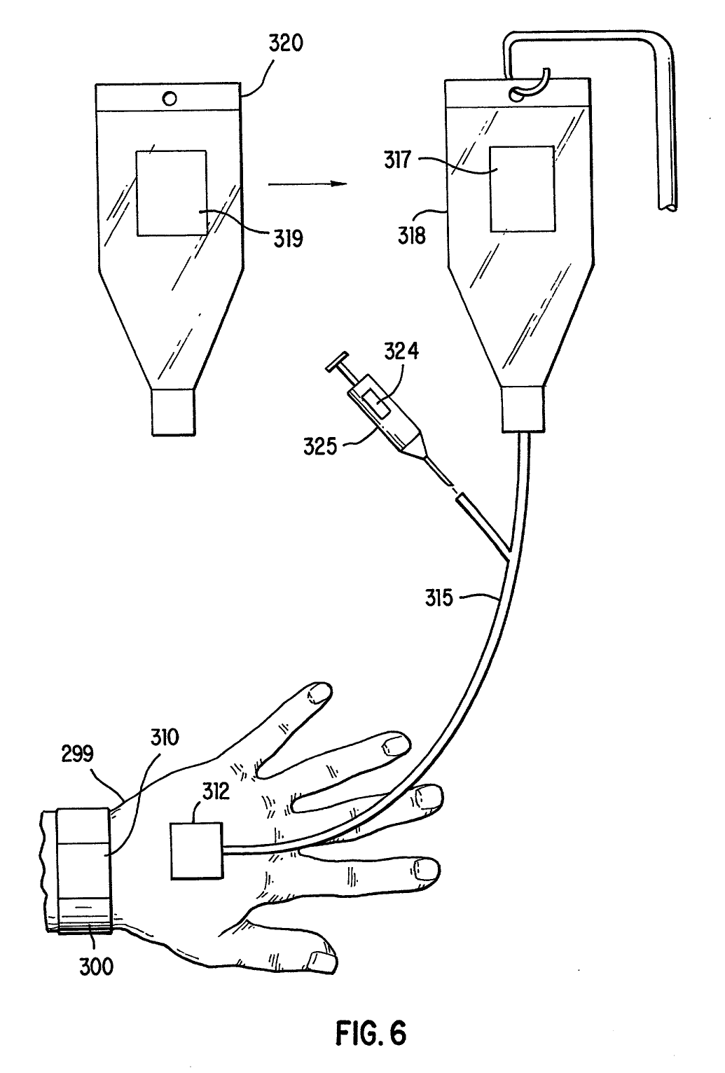

FIG. 6 shows another embodiment of the present invention. A patient 299 wearing an identification bracelet 300 is receiving fluids, medication, or blood 318, through tubing 315, intravenously 312. The identification bracelet 300 contains a sensor system 310, which includes information about the patient 299, including allergies, medical orders, medication orders, and the like. Each of the bags 318 and 320 include integrated circuits 317 and 319 respectively, which may be placed directly on the bags 318 and 320 or incorporated into a label and then placed on each bag 318 and 320. The integrated circuits 317 and 319 indicate what is in the bags, either blood, medication, fluids, etc. Likewise, syringe 325 contains medication and includes an integrated circuit 324, which indicates what medication is in the syringe 325. If the contents of bag 320 or syringe 325 are harmful, potentially harmful, or inappropriate in anyway for patient 299, then when the integrated circuits 319 or 324 come near the sensor system 310 located in the patient's identification bracelet 300, an alarm/alert (not shown) will sound. In an alternative embodiment, the sensor system can be located elsewhere in the patient's room. In addition, more than one integrated circuit can be located on or around the patient. In another embodiment one or more integrated circuits can be sensed by a sensor system and then the associated information from each integrated circuit is compared to the other or alternatively to stored information. If the information does not match a given set of parameters, an alert or alarm will sound.

In another embodiment of this invention, medical orders, such as for medical procedures, laboratory studies, or the like, are tagged with one or more integrated circuits-integral or removable, and a sensor system is located on or near the patient or in the patient record, card, chart, or hand held, or other computing platform. In another embodiment, the sensor system or sensor auxiliary device is located in the patient identification bracelet, dog tag, or other suitable appliance.

The patient sensor system is preprogrammed with patient information, including, for example, allergies, current medications, medical problem list, patient requests, consents, date of birth, name, insurance, next of kin, contact information, and the like, and may be programmed with status updates or orders. If an inappropriately tagged blood product or drug is brought in proximity to the patient, the sensor will trigger an alert or alarm which can take many forms for easy identification. Similarly, if a disposable integrated circuit card, for example, a 2″ by 3″ plastic card (i.e. credit card size) in which an integrated circuit was embedded, for each procedure is generated, should an orderly carrying this card approach the wrong patient for transport, an alert will be generated. The integrated circuit can be, for example a flash memory card or a smart card.

In another embodiment, a second integrated circuit can be located in the patient identification bracelet or dog tag. If both the medical orders and the patient identification bracelet contain integrated circuits, then the sensor system can monitor and track whether two integrated circuits move too close together. For example if the wrong medical orders were about to be placed in a patient's chart or the wrong medicine was to be given to a patient. In this embodiment, the sensor system can indicate a conflict between two integrated circuits visually or audibly. In addition, an output device, such as a monitor, can display which devices are in conflict.

In yet another embodiment of this invention, pharmaceutical products have one or more integrated circuits attached to the containers, bottles, bags, or labels which may be integral or removable for attachment to inventory lists, patient charts or intravenous (“IV”) or injection apparatus as noted above. Remote sensors on hand held devices, located in cabinets where pharmaceuticals are stored, or situated elsewhere, can quickly identify expired or misplaced or otherwise inappropriate drugs. Effective tracking of inventory with appropriate software is improved and appropriate ordering, billing and analysis of other information are enhanced.

In another embodiment of the present invention, a medical label includes at least one integrated circuit. The medical label can also be just the integrated chip. In addition, there can be more than one label on a given medical product. The medical label can be used to label any type of medical material or product, including pharmaceutical products and blood products, for example as shown in FIG. 6. The medical label can also be placed on medical containers, such as boxes, boxes that contain medical products, crates that contain medical products, bottles, ampoules, bags, syringes, or the like. The integrated circuit within the medical label can include information about the origination of the medical product, verification information about the medical product, the destination of the medical product, what the medical product is, which patient is to receive the medical product, indications, contra-indications, interactions, or similar medically or logistically relevant information. The verification information can include data that indicates the authenticity of the medical product. In addition, there can be more than one medical label on a given medical product. For example, an integrated circuit as described (either in a label or as the label itself) can be used and at least one additional label in the form of a written description of the medical product can be also located on the medical product.

In another embodiment where the medical label is used to label blood products, the integrated circuit can include collection, processing, storage, distribution, usage, and patient delivery information. Collection, processing, storage information, usage and the like can include, information about the blood donor, the blood type, blood recipient, expiration date, unit number, antigens, antibodies, logistical information, delivery distribution, or combinations thereof.

In addition, the label can have certain physical and chemical properties. For example, the label can be temperature resistant, water resistant, shock resistant, and flexible. The integrated circuit within the label can be hermetically sealed so that the environmental conditions experienced by the label do not effect the integrated circuit. For example, such environmental conditions can include the blood container containing the label being frozen and then thawed for storage purposes. The blood products referred to in these embodiments can include, but are not limited to, whole blood, platelets, packed red blood cells, and plasma.

### EXAMPLE

A patient is prepped for a surgical procedure and brought into the operating room. The operating room team comprising, for example, three operating room nurses, two doctors, and an anesthesiologist are also present in the operating room. The operating room nurses are responsible for, among other things, tracking the sponges, scalpels, gauze, forceps, clamps, and other medical implements used during the surgery or surgical procedure. To this end, each surgical implement to be used in this surgery includes an integrated circuit. As the nurses prepare for the surgery, they place each of the surgical implements on or near a sensor system, which is located near to the operating table upon which the patient lies. This sensor system registers each of the implements. As each of the implements is registered, the nurses watch the information appear on a screen of the sensor system, (e.g., a display of a computer) for each of the implements: 1) what each implement is; 2) the time the implement is placed on the sensor system; 3) the place where the implement is being registered; and 4) a unique identifier assigned to each implement is shown. Once all of the implements have been registered, the surgery can begin.

The doctors begin the surgery and each implement is used in turn. As each implement is used by the doctors, it is removed from the proximity of the sensor system. For example, when one of the nurses hands a scalpel to a doctor, the sensor system senses that the scalpel has been “checked-out” at a certain time. When the doctor has finished with the scalpel, a nurse can either put the scalpel back near the sensor platform it was removed from or place the scalpel on or near an auxiliary sensor system (e.g., a sharps container). When, for example, the auxiliary sensor system senses the scalpel, the scalpel is registered as “checked-in” and the location and time of check-in is also noted.

For each surgical implement, each of these steps can be performed. However, if at the end of the surgery, there are implements that have not been checked-in, then the sensor system indicates which implements are missing (e.g., not checked-in). In addition, prior to the doctors suturing the patient, a nurse checks the sensor system (e.g., the display of the computer mentioned earlier). In another embodiment, the sensor system can sound an alarm to remind the operating room team that there are implements missing.

Once the operating room team is aware that there are items missing and what items are in fact missing by looking at the information provided by the sensor system (e.g., the display of the computer again) as to the description of the item, the check-out time, and the like, a doctor can use an auxiliary sensor system in the form of a portable sensor system to locate the implement. For example, if the implement is still within the patient, a portable sensor system comparable to sensor system 100 but portable in nature is used to locate the missing implements.

Conclusion

Embodiments of devices, methods, systems to surgical implements and other medical products, including integrated circuits have been described. In the foregoing description, for purposes of explanation, numerous specific details are set forth to provide a thorough understanding of the present invention. It will be appreciated, however, by one skilled in the art that the present invention may be practiced without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the present invention.

In the foregoing detailed description, devices, systems and methods in accordance with embodiments of the present invention have been described with reference to specific exemplary embodiments. Accordingly, the present specification and figures are to be regarded as illustrative rather than restrictive.

We claim:

1. A medical product comprising:
a medical product including at least one integrated circuit that uniquely identifies the medical product by a unique identifier.

2. The medical product of claim 1, wherein the medical product is a surgical implement.

3. The surgical implement of claim 2, wherein the unique identifier is an alphanumeric string.

4. The surgical implement of claim 2, wherein the surgical implement is a sponge, a scalpel, a scissor, or a needle.

5. The surgical implement of claim 2, further comprising a memory that stores the unique identifier, and an analog front-end connected to the memory, wherein the analog front-end receives the unique identifier and transmits the unique identifier.

6. A method for monitoring and tracking surgical implements, the method comprising:
identifying at least one surgical implement including an integrated chip, wherein each surgical implement is uniquely identified.

7. The method of claim 6, further comprising monitoring the location of the at least one surgical implement.

8. The method of claim 7, wherein monitoring further includes recording a time the at least one surgical implement is moved.

9. The method of claim 6, wherein identifying further comprises registering the at least one surgical implements.

10. The method of claim 6, wherein the identifying and monitoring are performed by a detector.

11. A method for monitoring and tracking medical products, the method comprising:
uniquely identifying at least one medical products by a unique identifier, each medical product including at least one integrated circuit having the unique identifier programmed therein; and
monitoring each medical product by its unique identifier.

12. The method of claim 11, further comprising locating the at least one medical product, by receiving the unique identifier from the integrated circuit.

13. The method of claim 11, further comprising outputting the unique identifier from the integrated circuit for the at least one medical product.

14. The method of claim 13, where the outputting comprises visually displaying or audibly reproducing the unique identifier with a sensor system receiving the unique identifier.

15. The method of claim 11, wherein the at least one medical product includes patient records, patient orders, laboratory studies, patient procedures or combination thereof.

16. A method for monitoring surgical implements in conjunction with a surgical procedure, the method comprising:
initializing at least one surgical implement wherein each surgical implement includes an integrated circuit;
registering the at least one surgical implement prior to a surgical procedure by programming a unique identifier in the integrated circuit; and
accounting for the at least one surgical implement at the completion of said surgical procedure by receiving the unique identifier from the at least one surgical instrument.

17. The method of claim 16, wherein the initializing occurs upon entering an operating theater.

18. The method of claim 16, wherein the accounting for each of the surgical instruments occurs in the operating theater.

19. The method of claim 16, further comprising detecting any of the at least one surgical implements that is missing.

20. The method of claim 19, wherein each of the at least one surgical implements emits a signal containing the unique identifier.

21. The method of claim 16, further comprising:
indicating that at least one of the surgical implements is missing; and
locating the at least one missing surgical implements.

22. The method of claim 16, wherein the at least one surgical implement further includes a radio opaque material.

23. The method of claim 16, wherein the at least one surgical implements comprise a scalpel, sponge, needle, scissor, or combinations there of.

24. A system for monitoring and tracking surgical implements comprising:
at least one surgical implement, each surgical implement including an integrated circuit that stores a unique identifier of the at least one surgical instrument; and
a detector that detects the at least one surgical implement by detecting the unique identifier from the integrated circuit.

25. The system of claim 24, further comprising an output device that displays identifying data about each of the at least one surgical implements based on the unique identifier.

26. The system of claim 24, further comprising at least one auxiliary detector that detects the unique identifier from the integrated circuit.

27. The system of claim 24, wherein the at least one surgical implement comprise sponges, scalpels, needles, scissors, or combinations thereof.

28. A system for monitoring and tracking surgical implements comprising:
at least one surgical implements, including at least one integrated circuit; and
a sensor for sensing the at least one surgical implements based on a signal received from the at least one integrated circuit.

29. The system of claim 28, further comprising an output device that displays identifying data about each of the at least one surgical implements based on the signal received from the integrated circuit in the at least one surgical implement.

30. The system of claim 28, further comprising at least one auxiliary detector that detects the unique identifier from the integrated circuit.

31. The system of claim 28, wherein the surgical implements are sponges, scalpels, needles, scissors, laparoscopic or video assisted instruments or material, or combinations thereof.

32. A system for monitoring surgical implements used in conjunction with a surgical procedure comprising:
at least one surgical implement comprising an integrated circuit, the integrated circuit associating a unique identifier with each of said surgical implements and emitting a signal containing the unique identifier;
a detector that detects the signal emitted by said at least one surgical implement; and
an output device to process information provided by said detector.

33. The system of claim 32, wherein the output device displays a location of the at least one surgical implements based on the received signal.

34. The system of claim 32, wherein the output device is a computer.

35. The system of claim 32, further comprising at least one auxiliary detector that detects the unique identifier from the integrated circuit.

36. The system of claim 32, further comprising software contained in the detector or output device including a relational database.

37. The system of 32, wherein the at least one surgical implement comprises scalpels, sponges, needles, scissors, laparoscopic or video assisted instruments or material, or combinations thereof.

38. A system for monitoring surgical implements used in conjunction with a surgical procedure comprising:
at least one surgical implement comprising an integrated circuit, the integrated circuit associating a unique identifier with each of said surgical implements and emitting a signal containing the unique identifier;
a platform with a detector that detects the signal and determines a placement and removal of each of said surgical implements from said platform based on the detected signal; and
an output device that receives and processes information provided by said detector.

39. The system of claim 38, wherein the platform with a detector means is capable of detecting the time of placement and removal of the at least one surgical implements from said platform.

40. The system of claim 38, wherein the output device is a computer.

41. The system of claim 38, wherein the output device further comprises software including a relational database.

42. The system of claim 38, further comprising at least one auxiliary detector for detecting the location of each surgical implement.

43. The system of 38, wherein the at least one surgical implement comprises scalpels, sponges, needles, scissors, or combinations thereof.

44. A system for monitoring patients comprising:
at least one medical product, each medical product including a first integrated circuit;
at least one patient identification tag, each patient identification tag including a second integrated circuit, and
a sensor that monitors the medical products and patient identification tags based on signals received from the first and second integrated circuits.

45. The system of claim 44, further comprising an output device that indicates a conflict between a proximity of the at least one medical product to the at least one patient identification tag.

46. The system of claim 44, wherein the output device indicates the conflict visually or audibly.

47. The system of claim 44, further comprising auxiliary sensors that sense the unique identifier from the integrated circuit.

48. The system of claim 44, wherein the sensor or the auxiliary sensors sense when a medical product and a patient identification tag are in conflict.

49. A medical label comprising:
a label including at least one integrated circuit, wherein the integrated circuit uniquely identifies a medical product the medical label is attached to.

50. The medical label of claim 49, wherein the medical products are pharmaceutical containers.

51. The medical label of claim 49, wherein the pharmaceutical containers include boxes, crates, bottles, ampoules, bags, syringes, or combinations thereof.

52. The medical label of claim 49, wherein the integrated circuit contains medically or logistically relevant data.

53. The medical label of claim 52, wherein the medically or logistically relevant data includes, verification data, expiration date, unit number, antigens, antibodies, logistical information, delivery distribution, indications, contra-indications, interactions, or combinations thereof.

54. The medical label of claim 53, wherein the verification data verifies the authenticity of the medical product.

55. A blood product label comprising:
a label attached to a blood product, the label including at least one integrated circuit that uniquely identifies the blood product.

56. The blood product label of claim 55, wherein the label is temperature resistant.

57. The blood product label of claim 55, wherein the label is water resistant.

58. The blood product label of claim 55, wherein the label is shock resistant.

59. The blood product label of claim 55, wherein the label is flexible.

60. The blood product label of claim 55, wherein the at least one integrated circuit includes medically or logistically relevant data.

61. The blood product label of claim 60, wherein the medically or logistically relevant data includes, information about the blood donor, blood type, blood recipient, expiration date, unit number, antigens, antibodies, logistical information, delivery distribution, indications, contra-indications, interactions, or combinations thereof.

62. A blood product container including the blood product label of claim 55.

63. A medical product comprising:
at least one integrated circuit that uniquely identifies the medical product by a unique identifier.

64. The medical product of claim 63, wherein the medical product is a box containing medical products, a crate containing medical products, a bottle, an ampoule, a bag, a syringe, or combinations thereof.

65. The medical product of claim 63, wherein the medical product is a blood product.
